(19) United States
(12) Patent Application Publication
(10) Pub. No.: US 2026/0273276 A1
(43) Pub. Date: Sep. 17, 2026

(54) COLLABORATIVE-INTERVENTION CONTROL METHOD BASED ON BINAURAL FREQUENCIES AND TRANSCRANIAL DIRECT CURRENT STIMULATION

(71) Applicant: Shenzhen PanBrain Technology Co., Ltd., Shenzhen (CN)

(72) Inventors: Nailin YAO, Shenzhen (CN); Miaolong HE, Shenzhen (CN)

(73) Assignee: Shenzhen PanBrain Technology Co., Ltd., Shenzhen (CN)

(21) Appl. No.: 19/569,549

(22) Filed: Mar. 17, 2026

(30) Foreign Application Priority Data
Mar. 17, 2025 (CN) 202510313861.3

(51) Int. Cl. A61N 1/36; A61M 21/00; A61M 21/02

(52) U.S. Cl. CPC A61N 1/36025; A61M 21/02; A61N 1/36031; A61N 1/36034; A61M 2021/0027; A61M 2205/054; A61M 2205/3327; A61M 2205/50; A61M 2210/0687; A61M 2210/0693; A61M 2230/005; A61M 2230/10

(57) ABSTRACT

A collaborative-intervention control method based on binaural frequencies and transcranial direct current stimulation (tDCS) is provided, including steps of: controlling a preset intervention stimulation electrode to perform microcurrent stimulation during operation of a tDCS device; obtaining binaural frequencies, determining a frequency difference based on the binaural frequencies, and determining whether the frequency difference is within a preset target frequency range; and in response to the frequency difference being within the target frequency range, performing collaborative intervention based on the frequency difference and microcurrent output by the intervention stimulation electrode.

### CROSS-REFERENCE TO RELATED APPLICATIONS

The present disclosure claims priority to Chinese Patent Application No. 202510313861.3, filed on Mar. 17, 2025, the content of all of which is incorporated herein by reference.

### TECHNICAL FIELD

The present disclosure relates to the technical field of transcranial direct current stimulation (tDCS) device control, in particular to a collaborative-intervention control method based on binaural frequencies and tDCS.

### BACKGROUND

A tDCS device includes two types of electrodes: a detection electrode and a stimulation electrode. The detection electrode is used to collect an electroencephalogram (EEG) signal to evaluate a stimulation effect, while the stimulation electrode is used to intervene and stimulate a cerebral cortex. However, currently, when a user uses the tDCS device, the user generally relies solely on a single intervention method to perform microcurrent stimulation, and other methods of intervention stimulation are ignored. As a result, the intervention stimulation fails to achieve a desired effect, impacting user experience.

Therefore, the prior art needs to be improved and enhanced.

### SUMMARY

The present disclosure aims to provide a collaborative-intervention control method based on binaural frequencies and tDCS in view of the above-mentioned defects of the prior art. Technical solutions adopted by the present disclosure are as follows.

In a first aspect, the present disclosure provides a collaborative-intervention control method based on binaural frequencies and tDCS, including steps of:
controlling a preset intervention stimulation electrode to perform microcurrent stimulation during operation of a tDCS device;
obtaining binaural frequencies, determining a frequency difference based on the binaural frequencies, and determining whether the frequency difference is within a preset target frequency range; and
in response to the frequency difference being within the target frequency range, performing collaborative intervention based on the frequency difference and a microcurrent output by the intervention stimulation electrode.

In an implementation, the step of controlling a preset intervention stimulation electrode to perform microcurrent stimulation during operation of a tDCS device includes steps of:
obtaining initial electroencephalogram signal data before the operation of the tDCS device; and
activating the tDCS device, and controlling the intervention stimulation electrode to output the microcurrent with default stimulation information to achieve the microcurrent stimulation.

In an implementation, the step of controlling the intervention stimulation electrode to output the microcurrent with default stimulation information to achieve the microcurrent stimulation includes steps of:
obtaining a preset microcurrent intensity and stimulation duration of the intervention stimulation electrode, and using the preset microcurrent intensity and stimulation duration as the default stimulation information to achieve the microcurrent stimulation; or
obtaining a microcurrent intensity and stimulation duration of the intervention stimulation electrode during previous microcurrent stimulation, and using the microcurrent intensity and stimulation duration during the previous microcurrent stimulation as the default stimulation information to achieve the microcurrent stimulation.

In an implementation, the step of obtaining binaural frequencies, determining a frequency difference based on the binaural frequencies includes steps of:
outputting a left-channel acoustic-wave signal and a right-channel acoustic-wave signal respectively based on an acoustic-wave output component arranged at the tDCS device;
obtaining a first frequency corresponding to the left-channel acoustic-wave signal and a second frequency corresponding to the right-channel acoustic-wave signal respectively to obtain the binaural frequencies, where the first frequency is different from the second frequency; and
calculating a difference between the first frequency and the second frequency to obtain the frequency difference.

In an implementation, the target frequency range is 32 to 40 Hz.

In an implementation, the collaborative-intervention control method based on binaural frequencies and tDCS further includes steps of:
in response to the frequency difference being not within the target frequency range, adjusting the left-channel acoustic-wave signal and the right-channel acoustic-wave signal until the frequency difference is within the target frequency range.

In an implementation, the collaborative-intervention control method based on binaural frequencies and tDCS further includes steps of:
obtaining real-time electroencephalogram signal data during the collaborative intervention; and
evaluating an intervention effect based on the real-time EEG signal data, and based on the intervention effect, adjusting the microcurrent output by the intervention stimulation electrode and/or an acoustic-wave signal output by an acoustic-wave output component in real time.

In a second aspect, an embodiment of the present disclosure provides a collaborative-intervention control system based on binaural frequencies and tDCS configured to implement the steps of the collaborative-intervention control method based on binaural frequencies and tDCS, including:
a microcurrent stimulation module configured to control the preset intervention stimulation electrode to perform the microcurrent stimulation during the operation of the tDCS device;
a frequency-difference determination module configured to obtain the binaural frequencies, determine the frequency difference based on the binaural frequencies, and determine whether the frequency difference is within the preset target frequency range; and
a collaborative-intervention module configured to in response to the frequency difference being within the target frequency range, perform the collaborative intervention based on the frequency difference and the microcurrent output by the intervention stimulation electrode.

In a third aspect, an embodiment of the present disclosure provides a terminal device, including a memory, a processor, and a collaborative-intervention control program based on binaural frequencies and tDCS, where the collaborative-intervention control program based on binaural frequencies and tDCS is stored in the memory and executable on the processor, and when the processor executes the collaborative-intervention control program based on binaural frequencies and tDCS, the steps of the collaborative-intervention control method based on binaural frequencies and tDCS are implemented.

In a fourth aspect, an embodiment of the present disclosure provides a computer-readable storage medium, where a collaborative-intervention control program based on binaural frequencies and tDCS is stored in the computer-readable storage medium, and when a processor executes the collaborative-intervention control program based on binaural frequencies and tDCS, the steps of the collaborative-intervention control method based on binaural frequencies and tDCS are implemented.

Beneficial effects: Compared with the prior art, the present disclosure provides a collaborative-intervention control method based on binaural frequencies and tDCS, where the preset intervention stimulation electrode is controlled to perform the microcurrent stimulation during the operation of the tDCS device; then the binaural frequencies are obtained, the frequency difference is determined based on the binaural frequencies, and whether the frequency difference is within the preset target frequency range is determined; and then in response to the frequency difference being within the target frequency range, the collaborative intervention is performed based on the frequency difference and the microcurrent output by the intervention stimulation electrode. The present disclosure can perform intervention stimulation on a user through acoustic-current collaborative intervention, so as to influence a specific frequency band and enhance an intervention effect of the tDCS device.

### BRIEF DESCRIPTION OF DRAWINGS

FIG. 1 is a flowchart of an implementation of a collaborative-intervention control method based on binaural frequencies and tDCS provided by an embodiment of the present disclosure.

FIG. 2 is a functional-principle schematic diagram of the collaborative-intervention control method based on binaural frequencies and tDCS provided by an embodiment of the present disclosure.

FIG. 3 is a principle-block schematic diagram of a terminal device provided by an embodiment of the present disclosure.

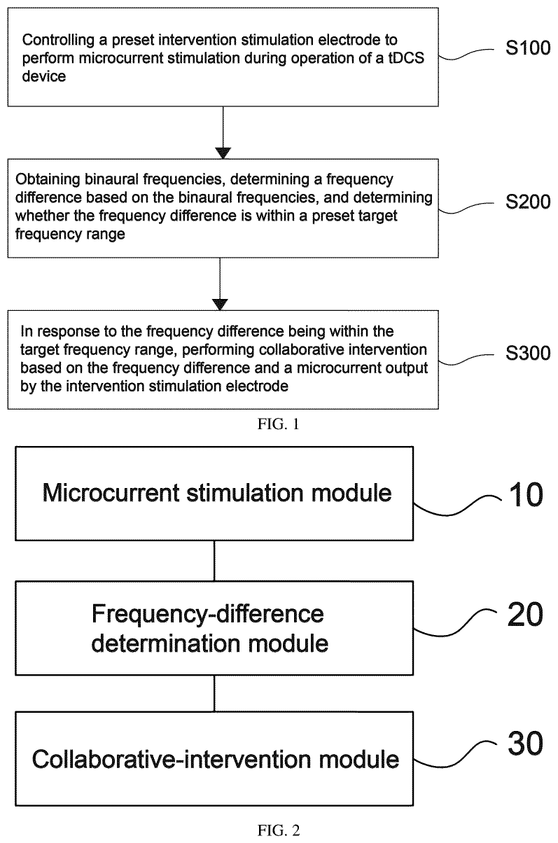

### DETAILED DESCRIPTION OF EMBODIMENTS

In order to make the purposes, technical solutions, and effects of the present disclosure clearer and more specific, the following provides further detailed explanations of the present disclosure with reference to the accompanying drawings and embodiments. It should be understood that the embodiments described herein are only for explaining the present disclosure and are not intended to limit the present disclosure.

In the present embodiment, intervention stimulation on a user can be achieved through acoustic-current collaborative intervention, so as to influence a specific frequency band and enhance an intervention effect of a tDCS device. During operation of the tDCS device in the present embodiment, firstly a preset intervention stimulation electrode is controlled to perform microcurrent stimulation. Then, binaural frequencies are obtained, a frequency difference is determined based on the binaural frequencies, and whether the frequency difference is within a preset target frequency range is determined. In response to the frequency difference being within the target frequency range, the collaborative intervention is performed based on the frequency difference and a microcurrent output from the intervention stimulation electrode.

A collaborative-intervention control method based on binaural frequencies and tDCS of the present embodiment may be applied to a terminal device, which may be an intelligent product terminal (e.g. a smartphone, a computer, etc.) connected to the tDCS device. During application, the terminal device of the present embodiment is connected to the tDCS device. The tDCS device includes an electroencephalogram (EEG) detection electrode and the intervention stimulation electrode. The EEG detection electrode is used to collect an EEG signal of the user. The intervention stimulation electrode delivers low current to stimulate an anterior temporal lobe and prefrontal cortex of a brain of the user. By stimulating the anterior temporal lobe and prefrontal cortex of the brain of the user, the tDCS device helps the user to keep attentive during work. Additionally, after performing the microcurrent intervention stimulation to the brain of the user, such as stimulating a dorsolateral prefrontal cortex (DLPFC) region, the tDCS device in the present disclosure can improve working memory and attention of the user. Furthermore, in addition to the intervention stimulation electrode, the tDCS device in the present disclosure is arranged with acoustic-wave output components, which are respectively located at a left side and right side of the tDCS device, respectively correspond to ear positions of the user, and are used to respectively output an acoustic-wave signal. Thus, the tDCS device in the present disclosure can output the left-channel acoustic-wave signal and the right-channel acoustic-wave signal, which have different frequencies, thereby generating the frequency difference. Research indicates that a specific acoustic-wave frequency affects a gamma (γ) wave of the brain, where the γ wave is generally associated with a higher-order cognitive function, e.g. attention and information processing. For example, 40 Hz auditory stimulation induces a γ-wave activity, which can be applied in Alzheimer's disease research to help clear amyloid proteins. Based on this, the present embodiment can perform the collaborative intervention on the user based on the frequency difference and the microcurrent generated by the intervention stimulation electrode, so as to stimulate a neuronal activity of the user, which effectively enhances a stimulation effect on the user, thereby improving the memory of the user and reducing the risk of the user developing Alzheimer's disease.

Based on the above, as shown in FIG. 1, the present embodiment provides a collaborative-intervention control method based on binaural frequencies and tDCS. The method of the present embodiment includes following steps.
Step S100: Controlling a preset intervention stimulation electrode to perform microcurrent stimulation during operation of a tDCS device.

The tDCS device in the present embodiment may be configured as a head-mounted device, similar to a helmet or a headband-style product. When a user wears the tDCS device, the user can press a start switch arranged on the tDCS device to issue a start command, so as to activate the tDCS device. An EEG detection electrode and intervention stimulation electrode in the tDCS device are located at different positions and are used to target different regions of a brain of the user, so as to stimulate corresponding regions of the brain, thereby helping the user maintain attention. During actual application, once the tDCS device is worn in place and activated, the tDCS device has not yet begun performing intervention stimulation on the user. At this time, in order to analyze an effect of the intervention stimulation in the present embodiment, EEG signal data from the brain of the user can be collected to obtain initial EEG signal data. The initial EEG signal data reflects original EEG data before the tDCS device begins to perform the intervention stimulation on the brain of the user, and the initial EEG signal data serves as reference data for determining whether a change has occurred in the EEG signal data.

In one implementation, after the tDCS device in the present disclosure is activated, a contact status between the EEG detection electrode and the brain of the user is collected. The contact status reflects a contact condition between the EEG detection electrode and the brain of the user, reflecting whether the EEG detection electrode is in close contact with the brain of the user, so as to avoid inaccurate detection of the EEG signal data due to poor contact. In actual application, the contact status between the EEG detection electrode and the brain of the user may be analyzed based on contact area or a contact pressure between the EEG detection electrode and the brain of the user. If the contact area between the EEG detection electrode and the brain of the user exceeds a preset area threshold, it indicates that the contact between the EEG detection electrode and the brain of the user is relatively close, and the contact status meets a preset requirement (i.e. the EEG detection electrode contacts the brain of the user in place). Similarly, if the contact pressure between the EEG detection electrode and the brain of the user exceeds a preset pressure threshold, it also indicates that the contact between the EEG detection electrode and the brain of the user is relatively close, and the contact status meets the preset requirement. When the contact status is determined to meet the preset requirement, the EEG detection electrode is triggered to be activated to obtain the initial EEG signal data. After the EEG detection electrode obtains the initial EEG signal data, the obtained initial EEG signal data is sent to a preset application (APP). Then the terminal device can record the initial EEG signal data based on the APP for a subsequent step involving corresponding analysis using the initial EEG signal data.

Next, the tDCS device in the present embodiment is activated and the intervention stimulation electrode is controlled to output the microcurrent with default stimulation information, so as to achieve the microcurrent stimulation. The default stimulation information in the present embodiment may be a preset microcurrent intensity and stimulation duration, or a microcurrent intensity and stimulation duration of the intervention stimulation electrode during previous microcurrent stimulation. Therefore, in the present embodiment, the preset microcurrent intensity and stimulation duration of the intervention stimulation electrode are obtained, and the preset microcurrent intensity and stimulation duration are used as the default stimulation information to achieve the microcurrent stimulation. Alternatively, the microcurrent intensity and stimulation duration of the intervention stimulation electrode during the previous microcurrent stimulation are obtained, and the microcurrent intensity and stimulation duration during the previous microcurrent stimulation are used as the default stimulation information to achieve the microcurrent stimulation.

In another implementation, the tDCS device in the present embodiment is provided with a mode selection menu, which includes various selectable intervention stimulation modes. In order to better assist the user in selecting the intervention stimulation modes, the present embodiment provides a mode recommendation to the user. In the present embodiment, a user login interface may be provided in the mode selection menu, where the user can input identity information of the user. After the tDCS device receives the identity information, the tDCS device can obtain historical usage data corresponding to the identity information. The historical usage data is data of the user using the tDCS device in a past period of time. Therefore, based on the historical usage data, the microcurrent intensity and stimulation duration of the intervention stimulation electrode during the previous microcurrent stimulation can be obtained, and then the microcurrent intensity and stimulation duration of the intervention stimulation electrode during the previous microcurrent stimulation can be used as the default stimulation information for performing intervention.

In another implementation, individual differences (such as differences in skull thickness and brain structure) may cause an insufficient effect of the microcurrent stimulation, thus the insufficient effect caused by the individual differences needs to be avoided to improve the effect of the microcurrent stimulation of the intervention stimulation electrode. Therefore, in the present embodiment, the default stimulation information may be adjusted based on the individual differences. In the present embodiment, based on magnetic resonance imaging (MRI) or computed tomography (CT) data provided in advance by the user, a skull-brain model is constructed using the MRI or CT data of the user, and an optimal electrode position and an optimal current intensity for the user are calculated. In this way, a terminal can make the intervention stimulation electrode in the tDCS device aligned with and contact the optimal electrode position, and adjust the microcurrent output by the intervention stimulation electrode to the optimal current intensity, thereby providing the optimal effect of the microcurrent stimulation for the user. In addition, in the present embodiment, the microcurrent stimulation may be applied to the user when the user engages in a specific cognitive task. For example, the user can perform a memory-training task simultaneously when the user uses the tDCS device, thereby using a neuroplasticity window to improve the effect of the microcurrent stimulation.
Step S200: Obtaining binaural frequencies, determining a frequency difference based on the binaural frequencies, and determining whether the frequency difference is within a preset target frequency range.

In order to achieve collaborative control, in the embodiment, the binaural frequencies are obtained, which include a first frequency corresponding to the left-channel acoustic-wave signal and a second frequency corresponding to the right-channel acoustic-wave signal. In the present embodiment, the left-channel acoustic-wave signal and the right-channel acoustic-wave signal may be output by the acoustic-wave output components arranged on the tDCS device. The first frequency and the second frequency in the present embodiment are different, thus a difference value between the first frequency and the second frequency can be determined as the frequency difference. In the present embodiment, it needs to determine whether the frequency difference is within the preset target frequency range. Research indicates that the specific acoustic-wave frequency has an impact on the γ wave of the brain, where the γ wave is generally associated with the higher-order cognitive function such as attention and information processing. For example, 40 Hz auditory stimulation induces the γ-wave activity, which can be applied in Alzheimer's disease research to help clear the amyloid proteins. However, due to the individual differences or as age increases, auditory stimulation below 40 Hz (e.g. 32 Hz) is more conducive to inducing the γ-wave activity. Based on this, in the present embodiment, the target frequency range may be set at 32 to 40 Hz. The target frequency range in the present embodiment is obtained by analyzing frontal EEG signals collected from users of different ages, so that the present embodiment can be applied to a wider user population. The collaborative intervention can be performed only when the frequency difference is within the target frequency range. Therefore, in the present embodiment, after obtaining the frequency difference, the frequency difference is compared with the target frequency range to determine whether the frequency difference is within the preset target frequency range.
Step S300: In response to the frequency difference being within the target frequency range, performing collaborative intervention based on the frequency difference and a microcurrent output by the intervention stimulation electrode.

When it is determined that the frequency difference is within the target frequency range, it is determined in the present embodiment that the frequency difference is within a range of 32 to 40 Hz at this time, and the auditory stimulation within this range can induce the γ-wave activity of the brain, which helps to clear the amyloid proteins. Therefore, the tDCS device can simultaneously output the left-channel acoustic-wave signal and the right-channel acoustic-wave signal through the acoustic-wave output components while outputting the microcurrent through the intervention stimulation electrode. Therefore, the user can be intervened and stimulated based on the microcurrent and the frequency difference, so as to stimulate the neuronal activity of the user, thereby effectively improving the effect of the stimulation on the user and reducing the risk of the user developing Alzheimer's disease while improving the memory of the user.

In the present embodiment, the left-channel acoustic-wave signal and the right-channel acoustic-wave signal are not fixed, and the collaborative intervention can be achieved as long as the frequency difference thereof is within the target frequency range. Therefore, in response to the frequency difference being not within the target frequency range, the left-channel acoustic-wave signal and the right-channel acoustic-wave signal are adjusted until the frequency difference is within the target frequency range. For example, by controlling the first frequency of the left-channel acoustic-wave signal to 160 Hz and the second frequency of the right-channel acoustic-wave signal to 200 Hz, the frequency difference can be controlled to 40 Hz. It can be seen that the left-channel acoustic-wave signal and the right-channel acoustic-wave signal can be adjusted according to actual needs. In the APP of the terminal device, a corresponding interface may be provided to adjust acoustic-wave frequencies (i.e. the first frequency and the second frequency) output by the acoustic-wave output components respectively located at left and right sides, so as to control the acoustic-wave output components respectively located at left and right sides to output the acoustic-wave signals with corresponding frequencies, so that the frequency difference is within the target frequency range. In another implementation of the present embodiment, the frequency difference is set directly in the APP of the terminal device. For example, in response to the frequency difference being set to 38 Hz, the APP automatically determines the acoustic-wave frequencies output by the acoustic-wave output components respectively located at left and right sides. For example, the first frequency of the left-channel acoustic-wave signal is controlled to be 208 Hz, and the second frequency of the right-channel acoustic-wave signal is controlled to be 170 Hz, so that the frequency difference between the two acoustic-waves is 38 Hz that is within the target frequency range.

In another implementation, in the present embodiment, real-time EEG signal data can be obtained during the collaborative intervention. Then, the intervention effect is evaluated based on the real-time EEG signal data, and the microcurrent output by the intervention stimulation electrode and/or the acoustic-wave signals output by the acoustic-wave output components are adjusted in real time based on the intervention effect. For example, when it is found that there is no significant difference between the real-time EEG signal data and the initial EEG signal data after the collaborative intervention (i.e. when there is no significant fluctuation in EEG before and after the intervention), real-time adjustment can be performed on the microcurrent stimulation of the intervention stimulation electrode and/or the acoustic-wave frequencies output by the acoustic-wave output components, thereby improving the intervention effect.

In summary, in the present embodiment, firstly, the preset intervention stimulation electrode is controlled to perform the microcurrent stimulation during the operation of the tDCS device; then, the binaural frequencies are obtained, the frequency difference is determined based on the binaural frequencies, and whether the frequency difference is within the preset target frequency range is determined; finally, in response to the frequency difference being within the target frequency range, the collaborative intervention is performed based on the frequency difference and the microcurrent output by the intervention stimulation electrode. In the present embodiment, the user is intervened and stimulated through the acoustic-current collaborative intervention to influence a specific frequency band and improve the intervention effect of the tDCS device.

Based on the above embodiment, the present disclosure further provides a tDCS device, which is used to implement the steps of the collaborative-intervention control method based on binaural frequencies and tDCS described in the above method embodiment. The tDCS device includes a microcurrent stimulation module 10, a frequency-difference determination module 20, and a collaborative-intervention module 30. The microcurrent stimulation module 10 is used to control a preset intervention stimulation electrode to perform microcurrent stimulation during operation of the tDCS device. The frequency-difference determination module 20 is used to obtain binaural frequencies, determine a frequency difference based on the binaural frequencies, and determine whether the frequency difference is within the preset target frequency range. The collaborative intervention module 30 is used to in response to the frequency difference being within the target frequency range, perform collaborative intervention based on the frequency difference and a microcurrent output by the intervention stimulation electrode.

In one implementation, the microcurrent stimulation module 10 includes:
an initial EEG signal obtaining unit, used to obtain initial EEG signal data before the operation of the tDCS device; and
a microcurrent stimulation output unit, used to activate the tDCS device and control the intervention stimulation electrode to output the microcurrent with default stimulation information, so as to achieve the microcurrent stimulation.

In one implementation, the microcurrent stimulation output unit includes:
a first microcurrent stimulation subunit, used to obtain a preset microcurrent intensity and stimulation duration of the intervention stimulation electrode, and use the preset microcurrent intensity and stimulation duration as the default stimulation information to achieve the microcurrent stimulation; or
a second microcurrent stimulation subunit, used to obtain a microcurrent intensity and stimulation duration of the intervention stimulation electrode during previous microcurrent stimulation, and use the microcurrent intensity and stimulation duration during the previous microcurrent stimulation as the default stimulation information to achieve the microcurrent stimulation.

In one implementation, the frequency-difference determination module 20 includes:
an acoustic-wave signal output unit, used to output a left-channel acoustic-wave signal and a right-channel acoustic-wave signal respectively based on an acoustic-wave output component arranged at the tDCS device;
a binaural-frequency determination unit, used to obtain a first frequency corresponding to the left-channel acoustic-wave signal and a second frequency corresponding to the right-channel acoustic-wave signal respectively to obtain the binaural frequencies, where the first frequency is different from the second frequency; and
a frequency-difference calculation unit, used to calculate a difference between the first frequency and the second frequency, so as to obtain the frequency difference.

In one implementation, the target frequency range is 32 to 40 Hz.

In one implementation, the tDCS device includes:
an acoustic-wave signal adjustment unit, used to in response to the frequency difference being not within the target frequency range, adjust the left-channel acoustic-wave signal and the right-channel acoustic-wave signal until the frequency difference is within the target frequency range.

In one implementation, the tDCS device further includes:
a real-time EEG signal obtaining unit, used to obtain real-time EEG signal data during the collaborative intervention; and
an intervention and adjustment unit, used to evaluate an intervention effect based on the real-time EEG signal data, and adjust the microcurrent output by the intervention stimulation electrode and/or an acoustic-wave signal output by the acoustic-wave output component in real time based on the intervention effect.

Working principles of each module/unit in the tDCS device in the present embodiment are the same as principles of each step in the above method embodiment, which are not repeated herein.

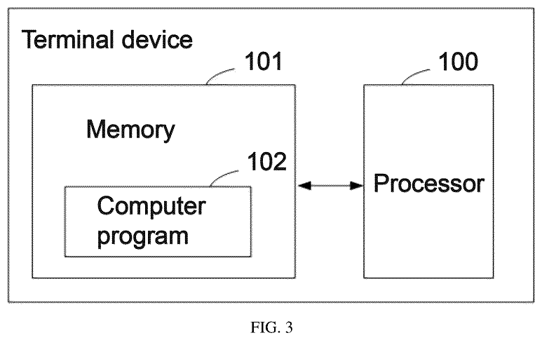

Based on the above embodiments, the present disclosure further provides a terminal device, and a principle-block schematic diagram of the terminal device is shown in FIG. 3. The terminal device may include one or more processors 100 (only one is shown in FIG. 3), a memory 101, and a computer program 102 that is stored in the memory 101 and executable on the one or more processors 100, and the computer program 102 is a collaborative-intervention control program based on binaural frequencies and tDCS. When the one or more processors 100 execute the computer program 102, various steps of the collaborative-intervention control method based on binaural frequencies and tDCS of the method embodiment can be implemented. Alternatively, when the one or more processors 100 execute the computer program 102, functions of each module/unit in the tDCS device of the device embodiment can be achieved without limitations herein.

In one embodiment, the processor 100 may be a central processing unit (CPU) or other general-purpose processors. The processor 100 may be a digital signal processor (DSP), an application-specific integrated circuit (ASIC), a field-programmable gate array (FPGA) or other programmable logic devices, a discrete gate or a transistor logic device, or a discrete hardware component. The general-purpose processor may be a microprocessor or may be any conventional processor.

In one embodiment, the memory 101 may be an internal storage unit of an electronic device, such as a hard drive or memory of the electronic device. The memory 101 may also be an external storage unit of the electronic device, such as a plug-in hard drive, smart media card (SMC), secure digital (SD) card, flash card, etc. equipped on the electronic device. Furthermore, the memory 101 may also include both the internal storage unit of the electronic device and the external storage unit. The memory 101 may be used to store a computer program and other programs and data required by the terminal device. The memory 101 may also be used to temporarily store data output or data to be output.

Those skilled in the art can understand that the principle-block schematic diagram shown in FIG. 3 is only a partial-structure block diagram related to the present disclosure, and does not constitute a limitation on the terminal device to which the present disclosure is applied. A specific terminal device may include more or fewer components than shown in the figure, or combine certain components, or have different component arrangements.

Those skilled in the art can understand that implementing all or part of the method steps in the above embodiments may be achieved by instructing a relevant hardware through a computer program. The computer program may be stored in a non-volatile computer-readable storage medium. The computer program when executed may include the method steps of the above embodiments. Any reference to memories, storage, operational databases, or other mediums used in the embodiments provided by the present disclosure may include a non-volatile and/or volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. By way of illustration, and not limitation, the RAM is available in various forms such as static RAM (SRAM), dynamic RAM (DRAM), synchronous DRAM (SDRAM), double data rate SDRAM (DDRSDRAM), enhanced SDRAM (ESDRAM), Synchlink DRAM (SLDRAM), Rambus direct RAM (RDRAM), direct Rambus dynamic RAM (DRDRAM), Rambus dynamic RAM (RDRAM), etc.

Finally, it should be noted that the above embodiments are only used to illustrate the technical solutions of the present disclosure, and not to limit the present disclosure. Although the present disclosure is described in detail with reference to the aforementioned embodiments, those skilled in the art should understand that they can still modify the technical solutions described in the aforementioned embodiments, or equivalently substitute a part of the technical features in the technical solutions. These modifications or substitutions do not make their corresponding technical solutions depart from the essence and scope of the embodiments of the present disclosure.

What is claimed is:

1. A collaborative-intervention control method based on binaural frequencies and transcranial direct current stimulation (tDCS), the method comprising steps of:
controlling a preset intervention stimulation electrode to perform microcurrent stimulation during operation of a tDCS device;
obtaining binaural frequencies, determining a frequency difference based on the binaural frequencies, and determining whether the frequency difference is within a preset target frequency range; and
in response to the frequency difference being within the target frequency range, performing collaborative intervention based on the frequency difference and a microcurrent output by the intervention stimulation electrode.

2. The collaborative-intervention control method based on binaural frequencies and tDCS according to claim 1, wherein the step of controlling a preset intervention stimulation electrode to perform microcurrent stimulation during operation of a tDCS device comprises steps of:
obtaining initial electroencephalogram signal data before the operation of the tDCS device; and
activating the tDCS device, and controlling the intervention stimulation electrode to output the microcurrent with default stimulation information to achieve the microcurrent stimulation.

3. The collaborative-intervention control method based on binaural frequencies and tDCS according to claim 2, wherein the step of controlling the intervention stimulation electrode to output the microcurrent with default stimulation information to achieve the microcurrent stimulation comprises steps of:
obtaining a preset microcurrent intensity and stimulation duration of the intervention stimulation electrode, and using the preset microcurrent intensity and stimulation duration as the default stimulation information to achieve the microcurrent stimulation; or
obtaining a microcurrent intensity and stimulation duration of the intervention stimulation electrode during previous microcurrent stimulation, and using the microcurrent intensity and stimulation duration during the previous microcurrent stimulation as the default stimulation information to achieve the microcurrent stimulation.

4. The collaborative-intervention control method based on binaural frequencies and tDCS according to claim 1, wherein the step of obtaining binaural frequencies, determining a frequency difference based on the binaural frequencies comprises steps of:
outputting a left-channel acoustic-wave signal and a right-channel acoustic-wave signal respectively based on an acoustic-wave output component arranged at the tDCS device;
obtaining a first frequency corresponding to the left-channel acoustic-wave signal and a second frequency corresponding to the right-channel acoustic-wave signal respectively to obtain the binaural frequencies, wherein the first frequency is different from the second frequency; and
calculating a difference between the first frequency and the second frequency to obtain the frequency difference.

5. The collaborative-intervention control method based on binaural frequencies and tDCS according to claim 1, wherein the target frequency range is 32 to 40 Hz.

6. The collaborative-intervention control method based on binaural frequencies and tDCS according to claim 4, further comprising steps of:
in response to the frequency difference being not within the target frequency range, adjusting the left-channel acoustic-wave signal and the right-channel acoustic-wave signal until the frequency difference is within the target frequency range.

7. The collaborative-intervention control method based on binaural frequencies and tDCS according to claim 1, further comprising steps of:
obtaining real-time electroencephalogram signal data during the collaborative intervention; and
evaluating an intervention effect based on the real-time EEG signal data, and based on the intervention effect, adjusting the microcurrent output by the intervention stimulation electrode and/or an acoustic-wave signal output by an acoustic-wave output component in real time.

8. A tDCS device configured to implement the steps of the collaborative-intervention control method based on binaural frequencies and tDCS according to claim 1, comprising:
a microcurrent stimulation module configured to control the preset intervention stimulation electrode to perform the microcurrent stimulation during the operation of the tDCS device;
a frequency-difference determination module configured to obtain the binaural frequencies, determine the frequency difference based on the binaural frequencies, and determine whether the frequency difference is within the preset target frequency range; and
a collaborative-intervention module configured to in response to the frequency difference being within the target frequency range, perform the collaborative intervention based on the frequency difference and the microcurrent output by the intervention stimulation electrode.

9. A terminal device, comprising a memory, a processor, and a collaborative-intervention control program based on binaural frequencies and tDCS, wherein the collaborative-intervention control program based on binaural frequencies and tDCS is stored in the memory and executable on the processor, and when the processor executes the collaborative-intervention control program based on binaural frequencies and tDCS, the steps of the collaborative-intervention control method based on binaural frequencies and tDCS according to claim 1 are implemented.
